(19) United States
(12) Patent Application Publication
(10) Pub. No.: US 2017/0231798 A1
(43) Pub. Date: Aug. 17, 2017

(54) GARMENT FOR IMPROVING POSTURE

(71) Applicant: Eric Shin, Montebello, CA (US)

(72) Inventor: Eric Shin, Montebello, CA (US)

(21) Appl. No.: 15/043,342

(22) Filed: Feb. 12, 2016

(51) Int. Cl. A61F 5/02; A41B 1/08

(52) U.S. Cl. CPC A61F 5/02; A41B 1/08; A61F 5/026

(57) ABSTRACT

A posture correction garment is designed as a compression shirt that may be worn in connection with various activities, such as daily work, exercise, physical therapy, and everyday life and leisure activities. The shirt can include shoulder straps extending from a back portion of the shirt, extending over the shoulders, and removably attaching at a front, upper region of the shirt. The shirt further includes one or more torso straps, extending from a back portion of the shirt, about the user's torso, and removably attaching to a front, lower region of the shirt. Wearing the shirt, by itself or under additional clothing, can help improve one's posture and help correctly align a user's shoulders and spine.

### BACKGROUND OF THE INVENTION

The present invention relates to articles of clothing and, more particularly, to a posture correcting compression shirt that can be worn in connection with various activities.

Posture refers to the position of a person's body when standing or sitting. It is a result of reflexes, habits, and adaptive responses to anything that resists a person being more or less upright and functional. Posture can be affected by gravity, working conditions, anatomy, hobbies or athletics, and the like.

Poor posture is the posture that results from certain muscles tightening up or shortening while others lengthen and become weak which often occurs as a result of one's daily activities. After a time, poor posture feels normal and continues to regress further from correct posture. When poor posture feels normal, it becomes harder to correct because the muscle memory now stores the information needed for poor posture. Poor posture will continue and increase one's digression from normal posture the longer it is left uncorrected.

There are numerous risks associated with poor posture. If not addressed properly, one's posture can be further harmed and can lead to increasingly painful experiences. Any distress in the spine, as well as other parts of the body can be increased due to prolonged periods of poor posture. Poor posture can impede the ability of the lungs to expand. Posture, when correct, helps to increases one's ability to breathe, and allows muscles to work at optimum capacity.

Many products are available to address poor posture. Some of these devices cannot be worn for various tasks and some of these devices have been found to do more harm than good. Being aware of their posture, straightening their back, lifting their chest, rolling their shoulders back and rotating their pelvis so their stomach and behind are tucked in will help improve one's posture. However, unless posture is part of a person's sport or exercise, people will most likely have a hard time being consistent with these physical posture correcting exercises.

As can be seen, there is a need for a device to help correct a person's posture that is safe and can be effective, even when the person is performing various typical daily tasks.

### SUMMARY OF THE INVENTION

In one aspect of the present invention, a garment comprises an open-ended torso section; an open neck portion; a shoulder attachment fabric disposed on an upper front portion of the garment; a torso attachment fabric disposed along opposite sides of a front portion of the garment; a back fabric member extending from the open neck portion to the open-ended torso section; shoulder straps extending from opposite sides of the back fabric member, the shoulder straps configured to extend over a user's shoulders and attach to the shoulder attachment fabric; and at least one torso strap extending from each side of the back fabric member, the torso straps configured to extend about a user's torso and attach to the torso attachment fabric.

In another aspect of the present invention, a compression shirt comprises an open-ended torso section; an open neck portion; a shoulder attachment fabric disposed on an upper front portion of the garment; a back fabric member extending from the open neck portion to the open-ended torso section; and shoulder straps extending from opposite sides of the back fabric member, the shoulder straps configured to extend over a user's shoulders and attach to the shoulder attachment fabric.

In a further aspect of the present invention, a shirt comprises an open-ended torso section; an open neck portion; a shoulder attachment fabric disposed on an upper front portion of the garment; a torso attachment fabric disposed substantially vertically along opposite sides of a front portion of the garment; a back fabric member extending from the open neck portion to the open-ended torso section; shoulder straps extending from opposite sides of the back fabric member, the shoulder straps configured to extend over a user's shoulders and attach to the shoulder attachment fabric; and a pair of torso straps extending from each side of the back fabric member, the torso straps configured to extend about a user's torso and attach to the torso attachment fabric, wherein the shoulder straps and the at least one torso strap are formed from an elastic fabric; the torso attachment fabric and the shoulder attachment fabric include an outer surface having a hook retaining material; and at least ends of the shoulder straps and the at least one torso strap have an inner surface having a loop retaining material.

These and other features, aspects and advantages of the present invention will become better understood with reference to the following drawings, description and claims.

### BRIEF DESCRIPTION OF THE DRAWINGS

FIG. 1 is a perspective view of a posture correction garment according to an exemplary embodiment of the present invention;

FIG. 2 is a front view of the posture correction garment of FIG. 1;

FIG. 3 is a rear view of the posture correction garment of FIG. 1;

FIG. 4 is a front view of the posture correction garment of FIG. 1, illustrating sleeves that may be various lengths, such as short, three-quarter, or long, for example;

FIG. 5 is a front view of the posture correction garment of FIG. 1, illustrating unattached shoulder and torso straps; and

FIG. 6 is a front view of the posture correction garment of FIG. 1, illustrating the use of removable straps to assist a user in accessing the shoulder straps when the garment is worn.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a posture correction garment designed as a compression shirt that may be worn in connection with various activities, such as daily work, exercise, physical therapy, and everyday life and leisure activities. The shirt can include shoulder straps extending from a back portion of the shirt, extending over the shoulders, and removably attaching at a front, upper region of the shirt. The shirt further includes one or more torso straps, extending from a back portion of the shirt, about the user's torso, and removably attaching to a front, lower region of the shirt. Wearing the shirt, by itself or under additional clothing, can help improve one's posture and help correctly align a user's shoulders and spine.

As used herein, directional representations such as “vertical” and “horizontal” refer to directions when the shirt is worn by an erect-standing user.

For example, vertical may extend from a neck portion of the shirt toward the open-ended torso section of the shirt.

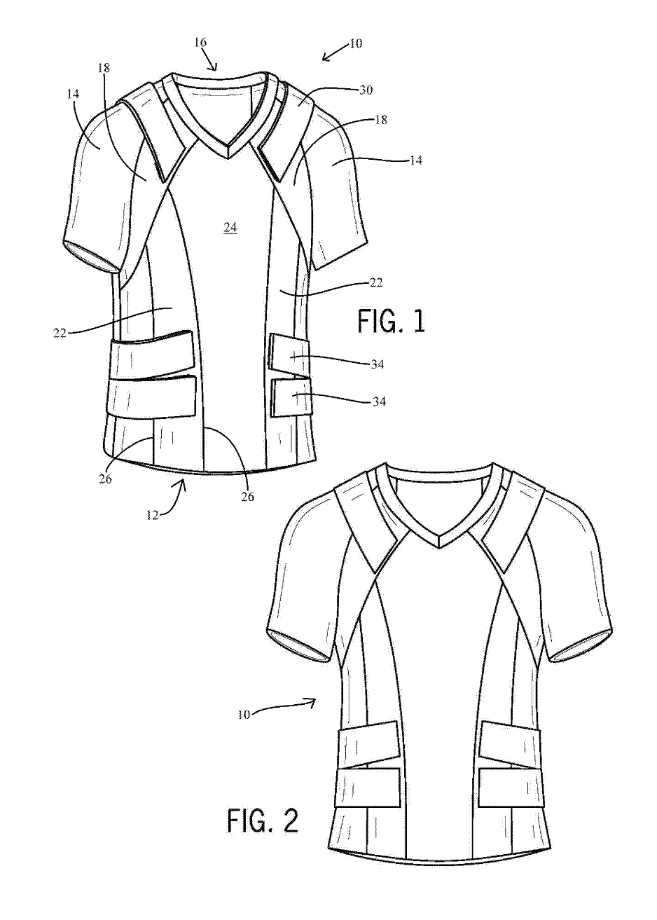

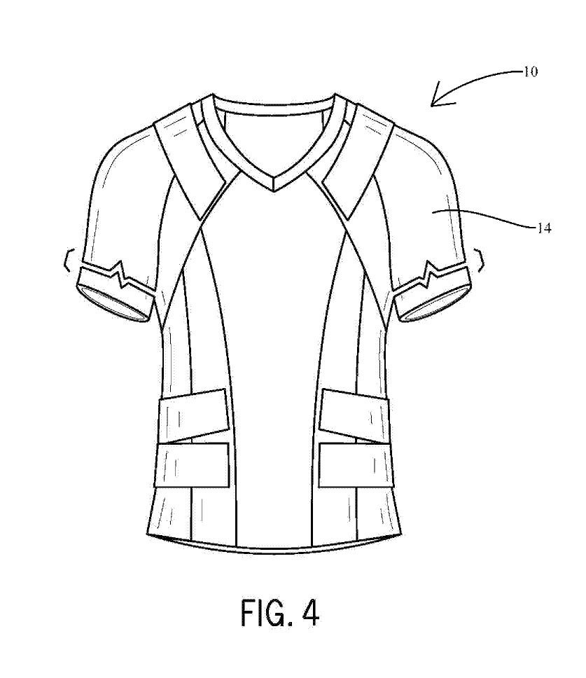

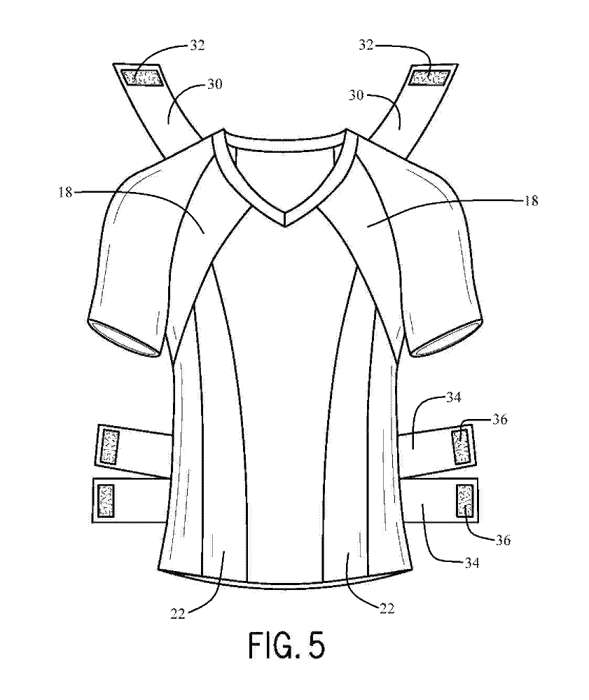

Referring now to FIGS. 1 through 5, a shirt 10 (also referred to as garment 10, posture correction garment 10 or compression shirt 10) typically includes an open-ended torso section 12, first and second arm sleeves 14 and a neck hole 16. The open-ended torso section 12 may be formed from or may be formed to include an elastic material adapted to elastically conform to a wearer's body during use of the shirt. For example, an elastic band may be stitched into the circumference of the opening formed in the of the open-ended torso section 12.

The sleeves 14, while shown as short sleeves, may vary in length to include sleeveless, short sleeves, three-quarter sleeves, long sleeves, or the like. Typically, the sleeves 14 are connected at a front portion of the shirt 10 at shoulder attachment fabric 18. The shoulder attachment fabric 18 may extend from the neck hole 16 to an underarm portion of the shirt 10. The sleeves 14 are typically connected at a rear portion of the shirt 10 at a back body 20. In some embodiments, the sleeves 14 may be raglan sleeves, where the material of the sleeve extends from the neck hole 16. Of course, other designs for sleeve formation and attachment are contemplated within the scope of the present invention.

The shirt 10 can also include a torso attachment fabric 22 attached to a front body 24 of the shirt 10. The shoulder attachment fabric 18 and the torso attachment fabric 22 may be made from the same or different material and is typically an elastic material having an exterior surface as a hook receptive fabric, such as a loop portion of a hook and loop fastener, for example. The torso attachment fabric 22 may extend essentially in a vertical configuration from the open-ended torso section 12 to the shoulder attachment fabric 18 disposed adjacent to the sleeves 14. In some embodiments, one or more princess seams 26 may be used to attach the torso attachment fabric 22 to the front body 24. Of course, other seam styles and various shirt cuts are contemplated within the scope of the present invention.

A vertical back fabric member 28 may extend from the neck portion 16 to the open-ended torso section 12. In some embodiments, the vertical back fabric member 28 may be made from a perforated mesh fabric, allowing ventilation for the user.

First and second shoulder straps 30 may attach to and extend from opposite sides of the vertical back fabric member 28, typically at an angle from about 5 degrees to about 45 degrees away from vertical. The shoulder straps 30 may be formed at least partially from an elastic material. Typically, the shoulder straps 30 are attached only at its end (at the vertical back fabric member 28), however, additional attachment may be made distal this attachment point.

A shoulder strap loop receptive fabric 32, such as a hook material of a hook and loop fastener, may be disposed on at least a portion of one side of the shoulder straps 30. While FIG. 5 show the loop receptive fabric 32 as being disposed at a portion of the end of the shoulder straps 30, the loop receptive fabric 32 may be incorporated on longer segments of the shoulder straps 30 to permit a greater range for size adjustment.

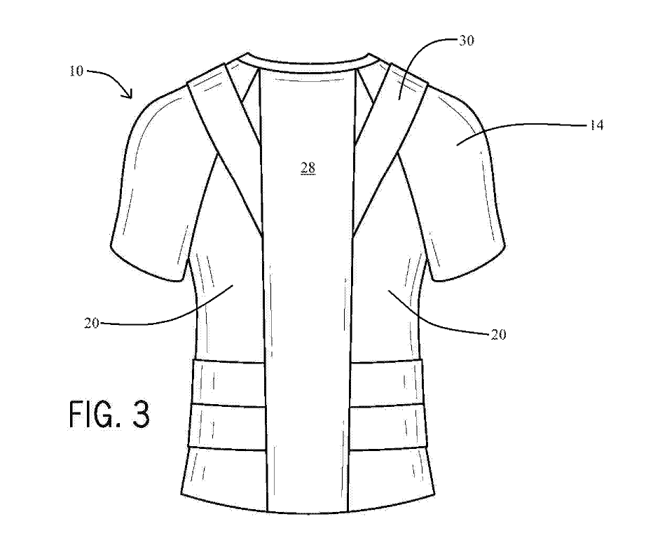

The shoulder straps 30 may be disposed over each shoulder of the wearer and may then attach, via a hook and loop attachment mechanism, to the shoulder attachment fabric 18, as shown in FIGS. 1 and 2, for example. While a hook and loop fastener system is shown, various other attachment mechanisms are contemplated within the scope of the present invention, such as a button attachment, a snap attachment, a buckle attachment or the like. The shoulder straps 30 are typically from about 1 inch to about 3 inches in width, typically about 2 inches in width. When in use, the shoulder straps 30 may help straighten a user's shoulders and help correct poor posture.

At least one torso strap 34 may attach to and extend from opposite sides of the vertical back fabric member 28. Typically, the torso straps 34 may extend from about 70 to about 110 degrees from vertical away from the vertical back fabric member 28. The torso straps 34 may be formed at least partially from an elastic material. Typically, the torso straps 34 are attached only at its end (at the vertical back fabric member 28), however, additional attachment may be made distal this attachment point. Typically, two torso straps 34 extend from each side of the vertical back fabric member 28. In some embodiments, the two torso straps 34 can at least partially overlap each other on each side of the shirt 10.

A torso strap loop receptive fabric 36, such as a hook material of a hook and loop fastener, may be disposed on at least a portion of one side of the torso straps 34. While FIG. 5 show the loop receptive fabric 36 as being disposed at a portion of the end of the torso straps 34, the loop receptive fabric 36 may be incorporated on longer segments of the torso straps 34 to permit a greater range for size adjustment.

The torso straps 34 may be disposed about the sides of the wearer and may then attach, via a hook and loop attachment mechanism, to the torso attachment fabric 22, as shown in FIGS. 1 and 2, for example. While a hook and loop fastener system is shown, various other attachment mechanisms are contemplated within the scope of the present invention, such as a button attachment, a snap attachment, a buckle attachment or the like. The torso straps 34 are typically from about 2 inches to about 5 inches in width, typically about 4 inches in width. When in use, the torso straps 34 may provide lumbar support for the user.

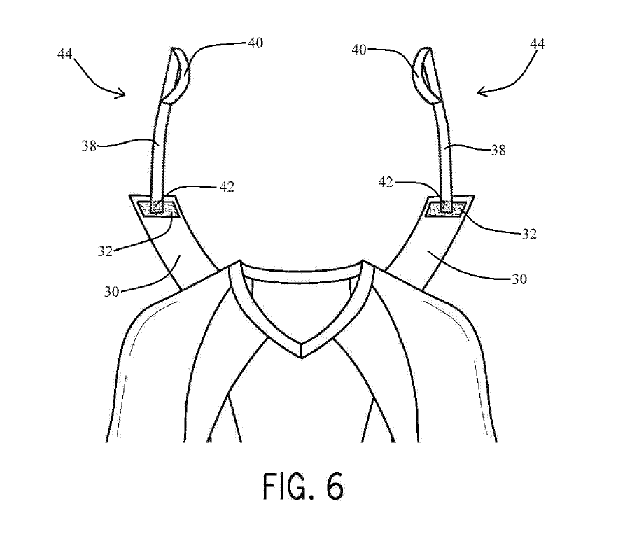

Referring now to FIG. 6, a strap positioning assistance member 44 may removably attach, at a first end 42 thereof, to the loop receptive fabric 32 of the shoulder straps 30 and extend therefrom as an elongated strap portion 38. The assistance member 44 may terminate at a fabric loop 40. The assistance member 44 may be placed on the shoulder straps 30 prior to a user wearing the shirt 10. The assistance member 44 then helps the user with access to the shoulder straps 30 and the positioning of the shoulder straps 30 over the user's shoulders. The assistance member 44 may be made of an elastic material, for example and may be formed of a suitable length to provide adequate assistance to the user. For example, the assistance member 44 may be from about 6 to about 24 inches in length.

The shirt 10 may be made of various materials as may be known in the art. In some embodiments, the majority of the shirt 10 may be made of synthetic material selected for moisture transport, high ventilation and heat dissipation. Such fabrics include, as non-limiting examples, polyester blends, nylon blends, and other such fabrics that wick moisture away from the skin to create a dry fit and touch.

It should be understood, of course, that the foregoing relates to exemplary embodiments of the invention and that modifications may be made without departing from the spirit and scope of the invention as set forth in the following claims.

1. A garment comprising:
an open-ended torso section;
an open neck portion;
a torso attachment fabric disposed along opposite sides of a front portion of the garment;
a back fabric member extending from the open neck portion to the open-ended torso section; and
at least one torso strap extending from each side of the back fabric member, the torso straps configured to extend about a user's torso and attach to the torso attachment fabric.

2. The garment of claim 1, wherein the at least one torso strap are formed from an elastic fabric.

3. The garment of claim 1, wherein the at least one torso strap includes two torso straps extending from each side of the back fabric member.

4. The garment of claim 1, wherein the torso attachment fabric include an outer surface having a hook retaining material.

5. The garment of claim 4, wherein at least ends of the at least one torso strap have an inner surface having a loop retaining material.

6. The garment of claim 1, wherein the shoulder attachment fabric extends from the open neck portion toward an underarm portion of the garment.

7. The garment of claim 6, further comprising sleeves attached to the garment.

8. The garment of claim 19, wherein the torso attachment fabric extends from the open-ended torso section to the shoulder attachment fabric.

9. The garment of claim 19, further comprising at least one assistance member removably attached and extending from an end of the shoulder straps.

10. A compression shirt comprising:
an open-ended torso section;
an open neck portion;
a shoulder attachment fabric disposed on an upper front portion of the garment;
a back fabric member extending from the open neck portion to the open-ended torso section; and
shoulder straps extending from opposite sides of the back fabric member, the shoulder straps configured to extend over a user's shoulders and attach to the shoulder attachment fabric.

11. The compression shirt of claim 10, further comprising:
a torso attachment fabric disposed along opposite sides of a front portion of the garment; and
at least one torso strap extending from each side of the back fabric member, the torso straps configured to extend about a user's torso and attach to the torso attachment fabric.

12. The compression shirt of claim 11, wherein the shoulder straps and the at least one torso strap are formed from an elastic fabric.

13. The compression shirt of claim 11, wherein the at least one torso strap includes two torso straps extending from each side of the back fabric member.

14. The compression shirt of claim 11, wherein the torso attachment fabric and the shoulder attachment fabric include an outer surface having a hook retaining material.

15. The compression shirt of claim 14, wherein at least ends of the shoulder straps and the at least one torso strap have an inner surface having a loop retaining material.

16. A shirt comprising:
an open-ended torso section;
an open neck portion;
a torso attachment fabric disposed substantially vertically along opposite sides of a front portion of the garment;
a back fabric member extending from the open neck portion to the open-ended torso section; and
a pair of torso straps extending from each side of the back fabric member, the torso straps configured to extend about a user's torso and attach to the torso attachment fabric, wherein
the at least one torso strap are formed from an elastic fabric;
the torso attachment fabric includes an outer surface having a hook retaining material; and
at least ends of the at least one torso strap have an inner surface having a loop retaining material.

17. The shirt of claim 20, further comprising sleeves attached to the shoulder attachment fabric at a front side of the garment.

18. The shirt of claim 20, further comprising at least one assistance member removably attached and extending from an end of the shoulder straps.

19. The garment of claim 1, further comprising:
a shoulder attachment fabric disposed on an upper front portion of the garment; and
shoulder straps extending from opposite sides of the back fabric member, the shoulder straps configured to extend over a user's shoulders and attach to the shoulder attachment fabric.

20. The shirt of claim 16, further comprising:
a shoulder attachment fabric disposed on an upper front portion of the garment; and
shoulder straps extending from opposite sides of the back fabric member, the shoulder straps configured to extend over a user's shoulders and attach to the shoulder attachment fabric.
